(19) United States
(12) Patent Application Publication
(10) Pub. No.: US 2013/0261015 A1
(43) Pub. Date: Oct. 3, 2013

(54) POLYPEPTIDE MARKERS FOR THE DIAGNOSIS OF CANCERS, AND METHODS FOR THE DIAGNOSIS OF CANCERS USING THE SAME

(71) Applicant: KOREA BASIC SCIENCE INSTITUTE, Daejeon (KR)

(72) Inventors: Yeong Hee AHN, Cheongju-si (KR); Jong Shin YOO, Seoul (KR); Park Min SHIN, Cheonwon-gun (KR)

(73) Assignee: Korea Basic Science Institute, Daejeon (KR)

(21) Appl. No.: 13/850,872

(22) Filed: Mar. 26, 2013

(30) Foreign Application Priority Data
Mar. 27, 2012 (KR) 10-2012-0031257

(51) Int. Cl. G01N 33/68

(52) U.S. Cl. CPC G01N 33/6893
USPC 506/9; 435/23; 435/7.1

(57) ABSTRACT

A method for diagnosing cancer using information on aberrant glycosylation of glycoproteins, which is related with cancer progression. More particularly, the present invention relates to a peptide marker for cancer diagnosis and a method for diagnosing cancer using the peptide marker, wherein glycoproteins aberrantly glycosylated due to cancer incidence and progression is isolated using lectin; and marker peptides generated by hydrolysis or the glycoproteins isolated by the lectin is selected and quantified.

### CROSS-REFERENCE TO RELATED APPLICATIONS

This patent application claims the benefit of priority from Korean Patent Application No. 10-2012-0031257, filed on Mar. 27, 2012, in the Korean Intellectual Property Office, the disclosure of which is incorporated herein by reference in its entirety.

### BACKGROUND OF THE INVENTION

1. Field of the Invention

The present disclosure relates to a method for diagnosing cancer by isolating and enriching glycoproteins which are aberrantly glycosylated due to cancer by using lectin, selecting polypeptides, and quantitatively analyzing the polypeptides.

2. Description of the Related Art

Protein glycosylation is one of the most representative post-translational modifications. When glycoproteins which are abundantly present on the surface of cell membranes receive a command of a specific signal, such as oncogenes, glycosylation occurs aberrantly. Many diseases have been known to correlate with aberrant actions of glycosyltransferases and glycolytic enzymes, which are caused by aberrant signal transduction of oncogenes. (Orntoft, T. F.; Vestergaard, E. M. Clinical aspects of altered glycosylation of glycoproteins in cancer. Electrophoresis 1999, 20:362-71).

Patterns of the aberrant glycosylation in cancer cells are very diverse, including increase in the site and the number of branches of N-linked glycan, increase in the sialylation and fucosylation, and changes in the glycan size, such as the formation of polylactosamine. With these phenomena proceeded in glycoproteins, these glycoproteins can be used for cancer markers for determining the existence and progression of cancer. Particularly, fucosylation which increases aberrantly in cancer cells, etc. provides the possibility to differentiate between proteins present in cancer cells and those in normal cells, and thus, glycoproteins which are glycosylated aberrantly can be developed as cancer markers for diagnosing cancer. After termination of required roles, glycoproteins which contain information on cancer are secreted into an extracellular media, or shed from the cellular membrane and released to a media, and therefore, culture media of various cancer cells, lysis products of cancer tissue, and particularly, blood of a patient can be suitable materials for detecting glycoproteins which contain information on cancer, that is, cancer markers.

In protein samples obtained from a normal group and a patient group, the differences in protein glycosylation can be an important cue for differentiating the patient group from the normal group, and therefore, many analytical methods to distinguish these differences have been developed. There are several methods which use the selectivity of lectin for the glycan structure of a glycoprotein to isolate and enrich glycoproteins or glycopeptides only, in order to distinguish the differences in glycosylation. ConA (Concanavalin A), WGA (Wheat germ agglutinin), Jacalin, SNA (Sambucas nigra agglutinin), AAL (Aleuria aurantia lectin), L-PHA (Phytohemagglutinin-L), PNA (Peanut agglutinin), LCA (Lens culimaris agglutinin-A), ABA (Agaricus biflorus agglutinin), DBA (Dolichos biflorus agglutinin), DSA (Datura stramonium agglutinin), ECA (Erythrina cristagalli agglutinin), SBA (Soybean agglutinin), SSA (Sambucas aeboldiana agglutinin), UEA (Ulex europaeus agglutinin), VVL (Vicia villosa lectin), BPL (Bauhinia purpurea lectin), or multilectin which uses mixtures of several lectins is used depending on the various glycan structures (Yang, Z. et al., J. Chromatogr, A, 2004, 1053, 79-88., Wang, Y. et al., Glycobiology, 2006, 16, 514-523). Since this method uses the selectivity of lectin for the glycan structures of glycoproteins, there is an advantage that the selective isolation and enrichment of glycoproteins having specific glycan structures is practicable. Particularly, by removing many proteins which do not exhibit the affinity for lectin through the process of isolating glycoproteins which are selective for lectin, the complexity of analysis samples can be significantly reduced. Isolated and enriched glycoproteins cam be analyzed qualitatively and quantitatively using various electrochemical methods, spectrochemical methods, and particularly, mass spectrometric methods.

Example of a method which has been mostly used is lectin-blotting method in which glycoprotein is analyzed using the selectivity of lectin for the glycan structure of the glycoprotein. In addition, the method has been generally used with immunoblotting method which shows high selectivity for specific proteins. Therefore, it is necessary to prepare an antibody against antigen glycoprotein and there is a disadvantage that the method cannot be used for proteins of which antibodies cannot be obtained. In addition, this lectin-blotting method which basically uses a gel-separation technology exhibits many limitations in the analysis speed, quantitative reliability, etc. Recently, antibody-lectin sandwich array method has been able to improve the analysis speed and analysis sensitivity significantly compared to the conventional lectin-blotting method (Forrester, S, et. al., Low-volume, high-throughput sandwich immunoassays for profiling plasma proteins in mice: identification of early-stage systemic inflammation in a mouse model of intestinal cancer, Mol Oncol 2007, 1(2):216-225). However, obtaining reliable antibody is necessary for this sandwich array method and obtaining antibodies for all glycoproteins which are being discovered on a large scale quickly is difficult.

Meanwhile, a mass spectrometric method is being used as an useful analysis method for a very high-speed and high-sensitivity qualitative and quantitative analysis of very complicated proteomic samples. Particularly, multiple reaction monitoring mass spectrometry (MRM MS) method provides a method which allows to quantify polypeptides of relatively small mass generated from proteolysis, quickly and with high reliability, and the method is particularly useful when antibody against the protein of interest cannot be obtained. MRM method is a high sensitivity quantitative analysis method, which allows the highly selective analysis of target peptides from very complicated samples, wherein the target peptides which are generated by proteolysis etc. of target proteins intended to be analyzed are isolated by one or more liquid chromatography followed by two stages of mass selection (precursor mass selection and fragment ion selection) (Andersen L, et al., Mol. Cell Proteomics. 2006, 5, 573-588).

Since the specimens such as plasma have widely varying concentrations of the proteins constituting the same, it is quite difficult to detect and quantitatively analyze a trace of plasma biomarker protein from the specimen which also contains high concentration proteins. Therefore, in order to discover plasma biomarkers of disease, removing high concentration proteins, such as albumin, immunoglobulin G (IgG), immunoglobulin A (IgA), transferring, haptoglobin, etc. which account for more than 90% of plasma to minimize complexity of clinical specimen and analyzing the remaining proteins may be preferable. When the concentration of the target marker protein in the clinical specimen is extremely low in spite of the minimization of sample complexity by removing high concentration plasma proteins and high selectivity for the target peptide by LC-MRM analysis, LOD (limit of detection) and LOQ (limit of quantification) for a cancer marker can be improved by enrichment of the marker protein using immunoaffinity, or by enrichment of hydrolyzed marker peptide.

Thus, the present inventors have found the fact that: hydrolyzed marker peptides derived from marker proteins causing cancer-specific glycosylation could be selected by isolating and enriching glycoproteins aberrantly glycosylated due to cancer by using lectin, hydrolyzing the glycoproteins to obtain polypeptides, and analyzing the polypeptides quantitatively; and cancer could be diagnosed by sequence analysis and a quantitative analysis of the marker peptides. The prevent inventors completed the present invention based on the fact.

### SUMMARY OF THE INVENTION

Exemplary embodiments of the present inventive concept overcome the above disadvantages and other disadvantages not described above. Also, the present inventive concept is not required to overcome the disadvantages described above, and an exemplary embodiment of the present inventive concept may not overcome any of the problems described above.

It is an object of the present invention to provide a method for cancer diagnosis, which use marker peptides which can trace a quantitative change in specific glycosylation of target glycoproteins following cancer incidence and progression.

It is another object of the present invention to provide a kit and a biochip for cancer diagnosis using the said marker peptides.

In order to achieve the object, the present invention provides a method for a quantitative analysis of polypeptides for providing information for cancer diagnosis, the method comprising the steps of: (1) isolating and enriching glycoproteins by treating a sample derived from a subject with lectin; (2) hydrolyzing the glycoproteins of step (1) to prepare glycoproteins-derived polypeptides; (3) performing a quantitative analysis on the polypeptides of step (2); and (4) if a polypeptide having any molecular weight selected from the group consisting of 685.4, 749.4, 778.4, 764.4, 851.5, 887.5, 921.4, 1007.5, 1014.6, 1057.5, 1075.6, 1089.6, 1109.5, 1189.6, 1575.8, 1640.9, 1754.9, 1778.8, 1803.0, 1332.9, 1855.0, 1890.8, 2056.9, 2258.1, 2573.3, 3180.6, and 3690.8 is detected as a result of the quantitative analysis of step (3), determining the subject as an individual who is more likely to have cancer or had cancer.

The present invention also provides a method for a sequence analysis and a quantitative analysis of polypeptides for providing information for cancer diagnosis, the method comprising the steps of: (1) isolating and enriching glycoproteins by treating a sample derived from a subject with lectin; (2) hydrolyzing the glycoproteins of step (1) to prepare glycoproteins derived polypeptides; (3) performing a sequence analysis and a quantitative analysis on the polypeptides of step (2); and (4) if a polypeptide having any amino acid sequence selected from the group consisting of SEQ ID NOs:1 to 27 is detected as a result of the sequence analysis and the quantitative analysis of step (3), determining the subject as an individual who is more likely to have cancer or had cancer.

Furthermore, the present invention provides a kit for cancer analysis comprising an antibody or a combination of antibodies which binds specifically to a polypeptide having any one of amino acid sequences of SEQ ID NOs:1 to 27.

The present invention also provides a biochip comprising biomolecules that are capable of binding specifically to a polypeptide having any one of amino acid sequences of SEQ ID NOs:1 to 27 and are accumulated on a solid substrate.

### ADVANTAGEOUS EFFECT

The present invention provides a method of distinguishing effectively between a normal group and a cancer patient group by analyzing quantitatively marker protein isoforms which have cancer-specific glycan structures which undergo quantitative changes in many kinds of cancer cells. The present invention can diagnose a cancer from a sample of a subject in an easy and quick manner by obtaining information on amounts of marker glycoprotein isoforms having cancer specific glycan structures through a quantitative analysis of marker peptides generated by hydrolysis. And the selected peptides can be useful for a marker for cancer diagnosis.

### BRIEF DESCRIPTION OF THE DRAWINGS

The above and/or other aspects of the present inventive concept will be more apparent by describing certain exemplary embodiments of the present inventive concept with reference to the accompanying drawings, in which:

FIG. 1 shows a result of MRM quantitative analysis of peptides samples obtained by the isolation and enrichment of the same amounts of pooled, cancer plasma and pooled control plasma using AAL (aleuria aurantia lectin) which has a selectivity for fucosylation followed by trypsin digestion.

FIG. 2 shows a box-and-whisker plot representing a result of MRM quantitative analysis of the marker peptide of SEQ ID NO:9 (molecular weight of the peptide, 1014.6 Da) using ten blood samples of hepatocellular carcinoma (HCC) patients and thirty control blood samples confirmed, that there were no cancer-related clinical findings.

FIG. 3 is a receiver operating characteristic (ROC) curve, representing a result of analyzing differentiations between ten liver cancer blood specimens and thirty control blood specimens, with respect to target peptide SEQ ID NO: 9.

FIG. 4 presents a result of MRM quantitative analysis additionally conducted on the peptides generated in the process of preparing specimen of marker peptide of SEQ ID NO: 9 from the other glycoproteins in the blood specimen, which are, alpha-1-acid glycoprotein 1 (A1AG1), alpha-1-antichymotrypsin (AACT), and ceruloplasmin (CERU).

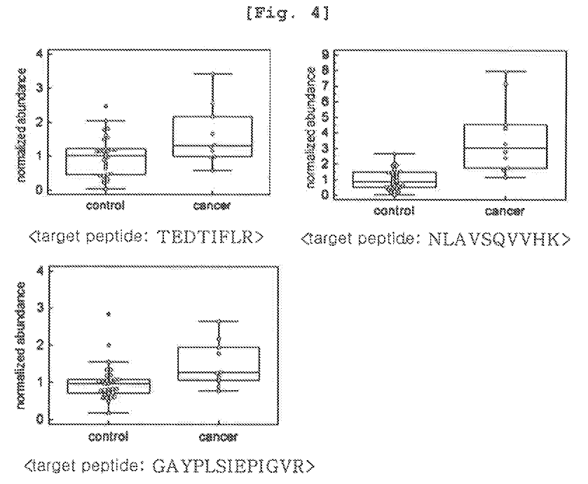

FIG. 5 presents a ROC curve wish respect to multi-markers, which are obtained by combining the result of quantitative analysis on the three target peptides originated from A1AG1, AACT and CERU with the result of quantitative analysis on the marker peptide of SEQ ID NO: 9, and statistically processing the resultant data.

### DESCRIPTION OF THE PREFERRED EMBODIMENTS

Features and advantages of the present intention will be more clearly understood by the following detailed description of the present preferred embodiments by reference to the accompanying drawings. It is first noted that terms or words used herein should be construed as meanings or concepts corresponding with the technical sprit of the present invention, based on the principle that the inventor can appropriately define the concepts of the terms to best describe his own invention. Also, it should be understood that detailed descriptions of well-known functions and structures related to the present invention will be omitted so as not to unnecessarily obscure the important point of the present invention.

Hereinafter, the present invention will be described in detail.

The present invention provides a method of diagnosis of cancer using information on specific glycosylation of glycoproteins.

Specifically, the present invention can diagnose cancer effectively by isolating glycoproteins which contain specific glycans involved in cancer incidence from a subject containing proteins using lectin; hydrolyzing the isolated glycoproteins to obtain peptides; selecting marker peptides from the obtained peptide samples by hydrolysis, wherein the marker peptides are capable of tracing a quantitative change in specifically glycosylated glycoproteins following cancer incidence; and using one or more the selected peptides as a marker.

The present invention provides a method for a quantitative analysis of polypeptides for providing information for cancer diagnosis, the method comprising the steps of:

(1) isolating and enriching glycoproteins by treating a sample derived from a subject with lectin;

(2) hydrolyzing the glycoproteins of step (1) to prepare glycoproteins derived polypeptides;

(3) performing a quantitative analysis on the polypeptides of step (2); and

(4) if a polypeptide having any molecular weight selected from the group consisting of 685,4, 749.4, 778.4, 784.4, 851.5, 887.5, 921.4, 1007.5, 1014.6, 1057.5, 1075.6, 1089.6, 1109.6, 1189.6, 1575.8, 1640.9, 1754,9, 1778.8, 1803.0, 1832.0, 1855.0, 1890.8, 2056.9, 2258.1, 2573.3, 3180.6, and 3690.8 is detected as a result of the quantitative analysis of step (3), determining the subject as an individual who is more likely to have cancer or had cancer.

The present invention also provides a method for a sequence analysis and a quantitative analysis of polypeptides for providing information for cancer diagnosis, the method comprising the steps of:

(1) isolating and enriching glycoproteins by treating a sample derived from a subject with lectin;

(2) hydrolyzing the glycoproteins of step (1) to prepare glycoproteins-derived polypeptides;

(3) performing a sequence analysis and a quantitative analysis on the polypeptides of step (2); and

(4) if a polypeptide having any amino acid sequence selected from the group consisting of SEQ ID NOs:1 to 27 is detected as a result of the sequence analysis and the quantitative analysis of step (3), determining the subject as an individual who is more likely to have cancer or had cancer.

In one embodiment, the subject of step 1) is a sample obtainable from an organism in which proteins which may contain information related with the existence and progressive state of cancer are present. Examples of subjects include biological tissues, cell lines or culture media established by biological tissue culture, saliva, blood, etc. After termination of required roles, glycoproteins which contain information on cancers are secreted into an extracellular media, or shed from the cellular membrane and released to a media, and therefore, particularly, culturing media of various cancer cell lines and blood of a patient are good specimens for detecting glycoproteins which contain information on cancers, that is, cancer markers. For a blood specimen, pretreatment to minimize sample complexity using a column for the removal of high concentration proteins [for example, MARS (multiple affinity removal system)], etc. may be performed because the concentration changes of component proteins present in blood are very great. However, more preferably, such sample pretreating process of removing high concentration proteins may be omitted, provided that there is no problem with sensitivity and reproducibility of target markers intended to be analyzed.

In one embodiment, the quantitative analysis on polypeptide of step 3) may preferably be accompanied with quantitative analysis on polypeptide originated from the hydrolysis of one or more glycoproteins selected from the group consisting of alpha-1-acid glycoprotein 1 (A1AG1), alpha-1-antichymotrypsin (AACT), and ceruloplasmin (CERU), but not limited thereto.

The polypeptide isolated with the hydrolysis of alpha-1-acid glycoprotein 1 may preferably be polypeptide with amino acid sequence expressed by SEQ ID NO: 28, the polypeptide isolated with the hydrolysis of alpha-1-antichymotrypsin (AACT) may preferably be polypeptide with amino acid sequence expressed by SEQ ID NO: 29, and the polypeptide isolated with the hydrolysis of alpha-1-ceruloplasmin (CERU) may preferably be polypeptide with amino acid sequence expressed by SEQ ID NO: 30, but not limited thereto.

In one embodiment, cancer incidence-related specific glycosylation of glycoproteins means that protein glycosylation are different from normal and occurs in cancer patients and those who experienced cancer. Such specific glycosylation may occur in glycans linked to glycosylation sites such as asparagines, threonine, or serine. Glycans haying cancer related specific structures share one glycosylation site with glycans having normal structures and exhibit glycan microheterogeneity. Therefore, the specific glycans exist as part of many glycan-isoforms present in one glycosylation site in a nonequivalently small amount as compared with the total amount of protein. In order to measure quantitative changes in specific glycans reliably, isolating these specific glycans from other glycan-isoforms having various structures and enriching them may be preferable, but not limited to such.

In one embodiment, lectin may be used in order to isolate and enrich an isoform having a specific glycan of interest from various glycan-isoforms having different glycan structures. Since this method uses the selectivity of lectin for the glycan structure of glycoproteins, it has an advantage that the selective isolation and enrichment of marker glycoproteins having specific glycan structures is practicable. Various lectins such as ConA, WGA, Jacalin, SNA, AAL, L-PHA, PNA, LCA, ABA, DBA, DSA, ECA, SBA, SSA, UEA, VVL, ox BPL, may be used alone or in combination depending on the structures of glycans to be isolated and enriched. In order to isolate proteins having glycan-isoforms of different structures selectively from the entire subject, various kinds of lectins may be selected to use.

In one embodiment, in order to trace a quantitative change in fucosylation which has been reported to increase in many kinds of cancer cells and cancer patients' blood, a glyco-isoform containing a glycan of fucose-structure was isolated and enriched using Aleuria aurantia lectin (AAL). Since many proteins which do not exhibit a selectivity for AAL are removed through the process of isolation of glycoproteins having a selectivity for AAL, the complexity of an analytical sample can be reduced significantly even without separate sample pretreatment process using the MARS, etc.

In one embodiment, high molecular weight proteins isolated by lectins may be hydrolyzed into lower molecular weight peptide fragments to improve analytical efficiency. For the step of hydrolyzing glycoproteins to obtain peptides, biological methods using various hydrolyzing enzymes or chemical methods using chemical reagents which are capable of inducing hydrolysis at specific amino acid site may be used. The hydrolyzing enzyme may be one or more hydrolyzing enzymes selected from the group consisting of Arg-C, Asp-N, Glu-C, Lys-C, chymotrypsin, and trypsin, and more preferably, but not limited to, trypsin. In the present invention, peptides of SEQ ID NOs:1 to 27 were taken into consideration as target peptides that are generated from glycoproteins enriched by lectins, if trypsin is used. However, if other kinds of hydrolyzing enzymes except for trypsin (for example, Arg-C, Asp-N, Glu-C, Lys-C, or chymotrypsin) are used, peptides of other sequences that may be generated from the same glycoproteins, including part of amino acid sequences of target peptides of SEQ ID NOs:1 to 27, of course, may be also taken into consideration as target peptides. For the hydrolysis efficiency and analytical efficiency for generated peptides, sample pretreatment processes such as denaturation, reduction, cysteine alkylation, etc. that are generally known may be performed prior to hydrolysis according to the need. Accordingly, peptides including cysteine of which mass was changed through such sample pretreatment process or methionine of which mass may be changed through oxidation process, of course, may also be taken into consideration as target peptides.

In one embodiment, the method may screen target peptide (marker peptides as an agent of a marker glycoprotein) which can trace a quantitative change in the glycosylated marker glycoprotein (Table 1), by quantitatively analyzing the hydrolyzed peptide specimens respectively obtained from the normal and patient groups. Culturing media from various cancer cell lines and patient's bloods particularly make good specimens for the detection of glycoproteins containing such cancer information (i.e., cancer markers). The cancer may include ail types of cancers that can induce cancer-specific glycosylation of the proteins, which may preferably include liver cancer, colorectal cancer, gastric cancer, lung cancer, uterine cancer, breast cancer, prostate cancer, thyroid cancer, and pancreatic cancer, and most preferably, liver cancer.

In one embodiment, in order to examine a quantitative change in fucosylated glycoproteins which increase in blood of a hepatocellular carcinoma patient, a quantitative analysis of candidate marker peptides which are agents of the glycoproteins are carried out and verified (refer to Table 1). In this way, a method for diagnosing cancer using peptide markers is provided according to the present invention.

In the present invention, marker peptides which are selected from peptide samples obtained by enrichment with lectins, followed by hydrolysis, may comprise one or more peptides derived from one glycoprotein, or comprise peptides derived from different glycoproteins (refer to Table 1). Therefore, two or more peptides together may be used as the selected marker peptides for a specimen analysis.

In the present invention, analytical methods based on immuno-precipitation/immune-blotting method which uses a selective antibody for peptides intended to be analyzed and mass analysis methods may used for the quantitative analysis method of hydrolyzed peptides containing marker peptides. Particularly, since mass analysis methods are free from the problem of the antibody obtainment for peptides intended to be analyzed, there is almost no limitation for target peptides which can be analyzed. Super high speed and high sensitivity analytical capability can also be strong points of mass analysis methods. Quantitative analysis methods by labeling peptides with isotope-labeled materials (iTRAQ, ICAT etc.) or quantitative analysis methods by adding an isotope-labeled standard (stable isotope standard) as an internal standard into a sample (multiple reaction monitoring, MRM), etc, may be used.

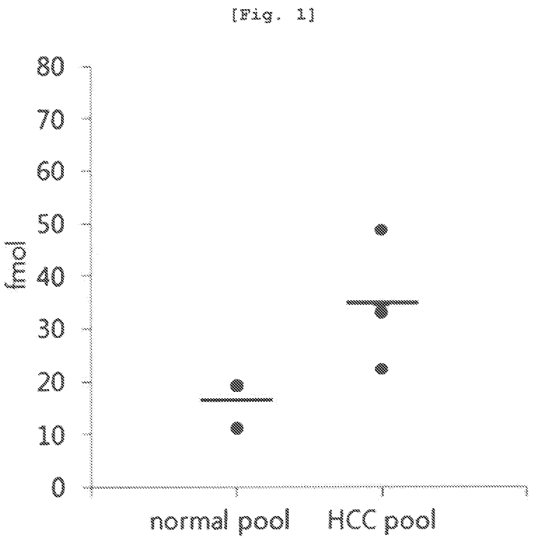

In a specific example of the present invention, it was investigated using blood samples enriched with AAL that whether a normal group and a HCC patient group can be differentiated using the marker peptide of SEQ ID NO:9 (molecular weight of the peptide: 1014.6) according to the present invention or not. Marker peptides were quantified by adding an isotope-labeled marker peptide standard as an internal standard to samples and performing MRM mass analysis. Specifically, HCC blood samples (pooled cancer plasma) were prepared by mixing blood samples of ten patients clinically confirmed to have HCC and normal comparative blood samples (pooled control plasma) were prepared by mixing blood samples of ten healthy people whose clinical findings are that there are no cancer-related diseases. Lectin-selective protein samples were isolated from the same amount of blood samples of the HCC patient group and the normal control group. Isolated samples were hydrolyzed, and peptide samples of the HCC patient group and the normal control group were obtained. Using the obtained peptide samples, the marker peptide of SEQ ID NO:9 was repeatedly quantified according to LC/MRM quantitative mass analysis method. Consequently, as shown in FIG. 1 the marker peptide was quantitated at a level of around 34.7 fmol in pooled cancer plasma and at a level of around 16.4 fmol in the same amount of pooled control plasma. That is, it was confirmed that the marker peptide of SEQ ID NO:9 of the HCC patient group was quantitated 2.1 times greater than that of the normal control group (refer to FIG. 1).

Based on the above result, the verification of peptide markers in the following Table 1 was carried out with real blood samples of HCC patients and people having no cancer-related findings according to the method of the present invention.

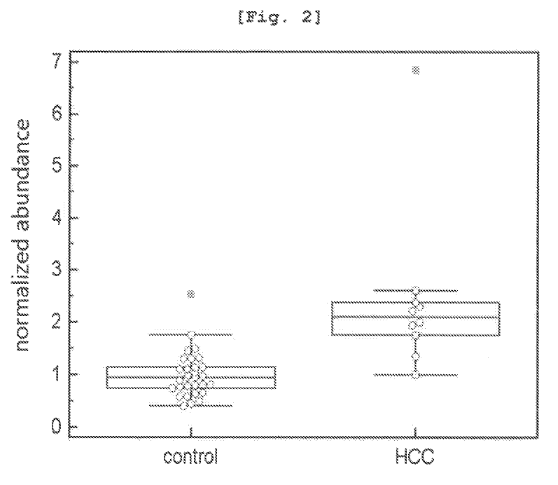

FIG. 2 is the result of MRM quantitative analysis of the marker peptide of SEQ ID NO:9 in each blood sample, wherein MRM quantitative analysis of the marker peptide of SEQ ID NO:9 was repeated three times using ten blood samples of hepatocellular carcinoma (HCC) patients confirmed clinically and thirty control blood samples confirmed that there were no cancer-related clinical findings, according to the method of the present invention. Thirty control blood samples consisted of ten samples having hepatitis B-infected findings, ten samples having hepatitis B-infected/cirrhosis findings, and ten samples having normal findings without the above two types of findings. The analysis measurements for the entire forty analyzed samples were normalized with the mean value obtained from control blood samples and shown in a box-and-whisker plot (refer to FIG. 2). It can be confirmed that the mean value of the marker peptide in ten HCC patients is twice or higher than the mean value of the marker peptide in thirty normal blood samples.

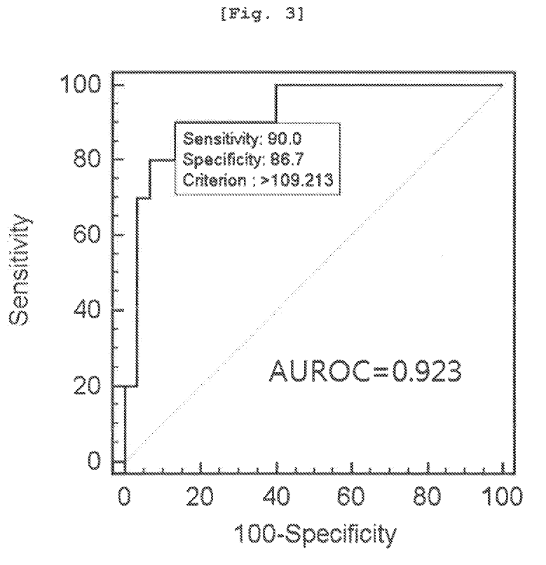

FIG. 3 shows an example of analyzing distinctiveness among ten liver cancer blood specimens and thirty blood specimens as a control used in FIG. 2, with Receiver Operating Characteristic (ROC) curve. With the area under ROC (AUROC)=0.923, the marker specificity is 86.7% at the sensitivity of 90%. From the above finding, it was verified that it is possible to distinguish between normal group and liver cancer patients based on the analysis on blood specimens using the marker peptide with SEQ ID NO: 9 (see FIG. 3).

Further, with the method of using peptide marker according to an embodiment, reliability of the developed marker glycoproteins will certainly be increased, if one or more marker peptides listed in Table 1 provided above, which can be generated with the hydrolysis of the same marker glycoproteins, are used in combination for the purpose of quantitative mass spectrometry. Furthermore, the MRM quantitative analysis on the peptides generated together from the other glycoproteins in the blood specimen in the process of producing marker peptide with SEQ ID NO: 9, which are, alpha-1-acid glycoprotein 1 (A1AG1), alpha-1-antichymotrypsin (AACT), and ceruloplasmin (CERU), was conducted along with the analysis on the marker peptide with SEQ ID NO: 9. That is, peptide TEDTIFLR with SEQ ID NO: 28 as a representative example of the cryptic peptides generated with hydrolysis of A1AG1, peptide NLAVSQVVHK with SEQ ID NO: 29 as a representative example of tryptic peptides generated with the hydrolysis of AACT, and peptide GAYPLSIEPIGVR with SEQ ID NO: 30 as a representative example of tryptic peptides generated with the hydrolysis of CERU, were selected and combined with peptide with SEQ ID NO: 9 and then went through quantitative analysis. As a result, it was confirmed that the peptides originated from the glycoproteins such as A1AG1, AACT, and CERU can also make marker peptides which can distinguish liver cancer specimens from the control specimens (see Table 4).

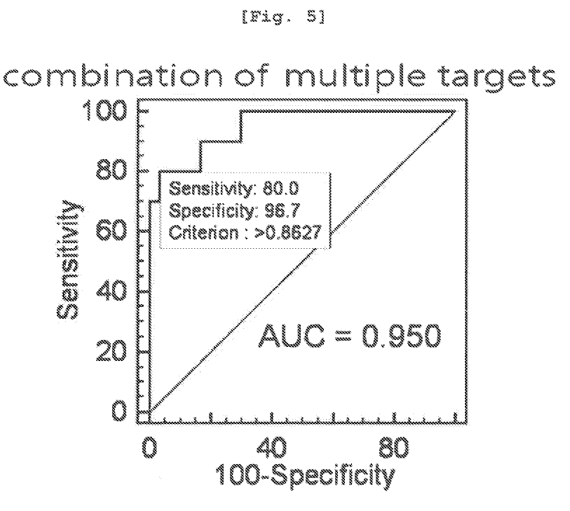

Additionally, the result of quantitative analysis on the three peptides TEDTIFLR, NLAVSQVVHK, GAYPLSIEPIGVR (SEQ IDS NOS: 28, 23, 30) as the representative examples of additional markers, were combined with the result of quantitative analysis on the marker peptide with SEQ ID NO; 9 using the artificial neural network algorithm, and the resultant data was statistically processed. FIG. 5 shows a ROC curve obtained as a result of combining the quantitative analysis results on the four marker peptides and statistically processing the combined data, in which the quantitative analysis results were obtained with respect to the ten liver cancer blood specimens and thirty blood specimens as a control. As a result, it was confirmed that the area under ROC (AUROC) obtained from the multi-markers based on the combination of the four individual peptide markers was 0.95, which was improved from the AUROC (0.92) obtained based on a single marker with SEQ ID NO: 9. From the above finding, it was verified that it is possible to distinguish between normal group and patients with liver cancer based on the analysis on blood specimens, using the marker peptide with SEQ ID NO: 9. It was also verified that, by additionally using the peptides originated from the glycoproteins such as A1AG1, AACT, and CERU as the multi-markers, it is possible to further increase the reliability of the method for distinguishing liver cancer specimens from the control specimens with no diagnosed liver cancer (see FIG. 5).

Therefore, it may be understood that the use of marker peptides which were discovered according to the present invention enables the differentiation between normal people and liver cancer patients from the analysis of blood specimens. In addition, it may be understood that the reliability of the marker glycoproteins of the present invention can be further improved by using, in combination, one or more marker peptides, which can be generated from the same marker glycoproteins by hydrolysis, or one or more marker peptides according to the present invention which can be generated by hydrolysis of different glycoprotein in which abnormal glycosylation occurs due to progress of cancer, for quantitative mass analysis.

Furthermore, the present invention provides a kit for cancer diagnosis comprising an antibody or a combination of antibodies which binds specifically to a polypeptide having any one of amino acid sequences of SEQ ID NOs: 1 to 27.

In the present invention, the molecular weight of the polypeptide having a amino acid sequences of SEQ ID NOs:1 is 685.4;

the molecular weight of the polypeptide having a amino acid sequences of SEQ ID NOs:2 is 749.4;

the molecular weight of the polypeptide having a amino acid sequences of SEQ ID NOs:3 is 778.4;

the molecular weight of the polypeptide having a amino acid sequences of SEQ ID NOs:4 is 784.4;

the molecular weight of the polypeptide having a amino acid sequences of SEQ ID NOs:5 is 851.5;

the molecular weight of the polypeptide having a amino acid sequences of SEQ ID NOs:6 is 887.5;

the molecular weight of the polypeptide having a amino acid sequences of SEQ ID NOs:7 is 921.4;

the molecular weight of the polypeptide having a amino acid sequences of SEQ ID NOs:8 is 1007.3;

the molecular weight of the polypeptide having a amino acid sequences of SEQ ID NOs:9 is 1014.6;

the molecular weight of the polypeptide having a amino acid sequences of SEQ ID NOs:10 is 1057.5;

the molecular weight of the polypeptide having a amino acid sequences of SEQ ID NOs:11 is 1075.6;

the molecular weight of the polypeptide having a amino acid sequences of SEQ ID NOs:12is 1089.6;

the molecular weight of the polypeptide having a amino acid sequences of SEQ ID NOs:13 is 1109.6;

the molecular weight of the polypeptide having a amino acid sequences of SEQ ID NOs:14 is 1189.6;

the molecular weight of the polypeptide having a amino acid sequences of SEQ ID NOs:15 is 1575.8;

the molecular weight of the polypeptide having a amino acid sequences of SEQ ID NOs:16 is 1640.9;

the molecular weight of the polypeptide having a amino acid sequences of SEQ ID NOs:17 is 1754.9;

the molecular weight of the polypeptide having a amino acid sequences of SEQ ID NOs:18 is 1778.8;

the molecular weight of the polypeptide having a amino acid sequences of SEQ ID NOs:19 is 1803.0;

the molecular weight of the polypeptide having a amino acid sequences of SEQ ID NOs:20 is 1832.9;

the molecular weight of the polypeptide having a amino acid sequences of SEQ ID NOs:21 is 1855.0;

the molecular weight of the polypeptide having a amino acid sequences of SEQ ID NOs:22 is 1890.8;

the molecular weight of the polypeptide having a amino acid sequences of SEQ ID NOs:23 is 2056.9;

the molecular weight of the polypeptide having a amino acid sequences of SEQ ID NOs:24is 2258.1;

the molecular weight of the polypeptide having a amino acid sequences of SEQ ID NOs:25 is 2573.3;

the molecular weight of the polypeptide having a amino acid sequences of SEQ ID NOs:26 is 3180.6; and

the molecular weight of the polypeptide having a amino acid sequences of SEQ ID NOs:27 is 3690.8.

The cancer may preferably be liver cancer, colorectal cancer, gastric cancer, lung cancer, uterine cancer, breast cancer, prostate cancer, thyroid cancer, and pancreatic cancer, and more preferably, liver cancer, but not limited thereto.

In the present invention, the kit can differentiate whether a subject has cancer or not by detecting a quantitative change in marker peptides which are generated from a sample of the subject by hydrolyzing enzyme treatment, and thus, it enables the monitoring, diagnosis, or screening of cancer.

In the present invention, the polypeptides, or the respective isotope-labeled peptides thereof may be additionally included as standard materials in the kit.

In the present invention, examples of antibody which can be used for the kit include polyclonal antibody, monoclonal antibody, fragments which can bind to an epitope, etc. The polyclonal antibody may be produced by a conventional method, wherein one or the peptide markers is injected to an animal, and blood is collected from the animal, and then serum containing antibodies is obtained. Such polyclonal antibody may be purified by any methods that are known in the art. The polyclonal antibody may be produced from any animal species host such as goats, rabbits, sheep, monkeys, horses, pigs, cattle, dogs, etc. The monoclonal antibody may be produced by any technology which provides the generation of antibody molecule through culture of continuous cell lines. Examples of such technologies include, but are not limited to, hybridoma technology, human B-cell hybridoma technology, and EBV-hybridoma technology (Kohler G et al., Nature 256:495-497, 1975; Kozbor D et al., J Immunol Methods 81:31-42, 1985; Cote R J et al., Proc Natl Acad Sci 80:2026-2030, 1983; and Cole S P et al., Mol Cell Biol 62:109-120, 1984). In addition, antibody fragments containing a specific binding site for any one of the peptide markers may be produced (Huse W D et al., Science 254: 1275-1281, 1989), As described above, the method of producing an antibody against a peptide having a specific sequence is obvious to a person skilled in the art.

In the present invention, the antibody which can be used for the kit may bind to a solid substrate in order to facilitate the following step, such as washing or complex isolation. Examples of the solid substrate include, but are not limited to, synthetic resin, nitrocellulose, glass substrate, metal substrate, glass fiber, microspheres, micro-beads, etc. Examples of the synthetic resin include, but are not limited to, polyester, polyvinyl chloride, polystyrene, polypropylene, PVDF, nylon, etc.

In the present invention, when the kit allows a sample obtained from a subject to contact with the antibody which can bind specifically to any one of the peptide markers bound to the solid substrate, the sample may be diluted to a suitable degree prior to the contact with the antibody.

In the present invention, the kit allows a sample obtained from a subject to contact with the antibody which can bind specifically to any one of the peptide markers bound to the solid substrate, and then additionally, proteins etc, which are not. bound to the antibody are washed to be removed, and then, the kit may detect marker peptides.

In the present invention, the kit may additionally comprise a detecting antibody which binds specifically to the peptide markers. The detecting antibody may be a conjugate labeled with a detector such as a color-developing enzyme, a fluorescent material, a radioisotope, or colloid, and preferably a secondary antibody which can bind specifically to the marker, but is not limited to such. The color-developing enzyme may be, but is not limited to, peroxidase, alkaline phosphatase, or acid phosphatase (for example, horseradish peroxidase), The fluorescent material may be, but is not limited to, fluorescein carboxylic acid (FCA), fluorescein isothiocyanate (FITC), fluorescein thiourea, (FCA), 7-acetoxycoumarin-3-yl, fluorescein-5- yl, fluorescein-6-yl, 2′,7′-dichlorofluroescein-5-yl, 2′,7′-dichlorofluroescein-6-yl, dihydrotetramethylrosamine-4-yl, tetramethylrhodamine-5-yl, tetrametylrhodamine-6-yl, 4,4-diflouro-5,7-dimethyl-4-bora-3a,4a-diaza-s-indacene-3-ethyl, or 4,4-difluoro-5,7-diphenyl-4-bora-3a,4a -diaza-s-indacene-3-ethyl.

In the present invention, the kit may additionally comprise a substrate to do the color development reaction with enzyme and a wash or eluent which can remove unbound proteins etc. and retain bound peptide markers only.

The present invention also provides a biochip for cancer diagnosis, the biochip comprising biomolecules that are capable of binding specifically to a polypeptide having any one of amino acid sequences of SEQ ID NOs:1 to 27 and are accumulated on a solid substrate.

In the present invention, the molecular weight of the polypeptide having a amino sequences of SEQ ID NOs:1 is 685.4;

the molecular weight of the polypeptide having a amino acid sequences of SEQ ID IDs:2 is 749.4;

the molecular weight of the polypeptide having a amino acid sequences of SEQ ID NOs:3 is 778.4;

the molecular weight of the polypeptide having a amino acid sequences of SEQ ID NOs:4 is 784.4;

the molecular weight of the polypeptide having a amino acid sequences of SEQ ID NOs:5 is 851.5;

the molecular weight of the polypeptide having a amino acid sequences of SEQ ID NOs:6 is 887.5;

the molecular weight of the polypeptide having a amino acid sequences of SEQ ID NOs:7 is 921.4;

the molecular weight of the polypeptide having a amino acid sequences of SEQ lb NOs:8 is 1007.5;

the molecular weight of the polypeptide having a amino acid sequences of SEQ ID NOs:9 is 1014.6;

the molecular weight of the polypeptide having a amino acid sequences of SEQ ID NOs:10 is 1057.5;

the molecular weight of the polypeptide having a amino acid sequences of SEQ ID NOs:11 is 1075.6;

the molecular weight of the polypeptide having a amino acid sequences of SEQ ID NOs:12 is 1089.6;

the molecular weight of the polypeptide having a amino acid sequences of SEQ ID NOs:13 is 1109.6;

the molecular weight of the polypeptide having a amino acid sequences of SEQ ID NOs:14 is 1189.6;

the molecular weight of the polypeptide having a amino acid sequences of SEQ ID NOs:15 is 1575.8;

the molecular weight of the polypeptide having a amino acid sequences of SEQ ID NOs:16 is 1640.9;

the molecular weight of the polypeptide having a amino acid sequences of SEQ ID NOs:17 is 1754.9;

the molecular weight of the polypeptide having a amino acid sequences of SEQ ID NOs:18 is 1778.8;

the molecular weight of the polypeptide having a amino acid sequences of SEQ ID NOs:19 is 1803.0;

the molecular weight of the polypeptide having a amino acid sequences of SEQ ID NOs:20 is 1832.9;

the molecular weight of the polypeptide having a amino acid sequences of SEQ ID NOs:21 is 1855.0;

the molecular weight of the polypeptide having a amino acid sequences of SEQ ID NOs:22 is 1890.8;

the molecular weight of the polypeptide having a amino acid sequences of SEQ ID NOs:23 is 2056.9;

the molecular weight of the polypeptide having a amino acid sequences of SEQ ID NOs:24 is 2258.1;

the molecular weight of the polypeptide having a amino acid sequences of SEQ ID NOs:25 is 2573.3;

the molecular weight of the polypeptide having a amino acid sequences of SEQ ID NOs:26 is 3180.6; and

the molecular weight of the polypeptide having a amino acid sequences of SEQ ID NOs:27 is 3690.8.

The polypeptide with one or more amino acid sequences from among SEQ ID NOS: 1 to 27 may preferably be polypeptide labeled with stable isotope, but not limited thereto.

The cancer may preferably be selected from the group consisting of liver cancer, colorectal cancer, gastric cancer, lung cancer, uterine cancer, breast cancer, prostate cancer, thyroid cancer, and pancreatic cancer, and more preferably, liver cancer, but not limited thereto.

In the present invention, the biochip can differentiate whether a subject has cancer or not by detecting a quantitative change in marker peptides which are obtained from a sample of the subject by hydrolyzing enzyme treatment, and thus, it enables the monitoring, diagnosis, and screening of cancer.

In the present invention, the biomolecule may be, but is not limited to, an antibody or an aptamer. The biomolecules mean organic molecules produced by a living organism, including small molecules, such as primary metabolites, secondary metabolites, and natural substances, as well as macropolymers, such as proteins, polysaccharides, and nucleic acids. The aptamer means an oligonucleotide or a peptide that binds to a specific target molecule.

In the present invention, the solid substrate may be selected from, the group consisting of plastic, glass, metal, and silicon, but is not limited to such.

Hereinafter, the present invention will be described in more detail with reference to the following examples.

However, the following examples are provided for illustrative purposes only, and the scope of the present invention should not be limited, thereto in any manner.

### EXAMPLE 1

### Sample Preparation

HCC blood samples (pooled cancer plasma) were prepared by mixing blood samples of ten patients clinically confirmed to have HCC and normal comparative blood samples (pooled control plasma) were prepared by mixing blood samples of ten healthy people whose clinical findings are that there are no cancer-related diseases. Using AAL (aleuria aurantia lectin) which shows a selective affinity for glycoproteins having a fucose glycan, AAL-selective protein samples were isolated from the same amount of blood samples of the HCC patient group and the normal control group. Isolated samples were hydrolyzed, and peptide samples of the HCC patient group and the normal control group were obtained. As for a support for fixing lectins, various kinds of supports, including agarose beads, magnetic beads, etc. may be used. For the analysis of the present clinical blood samples, strepavidine-magnetic beads were used for fixing lectins. That is, respective blood samples of the HCC patient group and the normal control group were added to AAL-biotin-strepavidine-magnetic beads under phosphate-buffered saline (PBS) and allowed to stand for 12 hr at 4° C. Lectin-bound proteins were washed three times with PBS, and then, proteins were detached from lectins with 2M urea/dithiothreitol (DDT) solution. Obtained proteins were treated with iodoacetamide (IAA), dilated two-fold with 50 mM ammonium bicarbonate, and then, hydrolyzed with trypsin for overnight at 37° C. The hydrolyzed peptides were dried under reduced pressure.

### EXAMPLE 2

### Selection of Candidate Markers by Peptide Analysis

In order to analyze samples prepared in the sample preparation in <Example 1>, LC/ESI-MS/MS was performed using HPLC (high-performance liquid chromatography; trap column: C18, 5 um, 300 um×5 mm; analytical column: C18, 5 um, 75 um×10 cm) tandem LTQ-FT mass spectrometer (Thermo Finnigan), the electrospray ionization (ESI) mass spectrometer. Part of peptide samples prepared by trypsin hydrolysis of each protein sample was diluted 10-fold and 10 μL aliquots were injected into HPLC/mass spectrometer.

Based on the mass analysis result, hydrolyzed peptides of proteins enriched by AAL can be confirmed through a search engine, such as MASCOT, SEQUEST, etc. Significant proteins were searched from the peptides obtained from LC/ESI-MS/MS analysis and analysis frequency thereof, etc. Glycosylation and cancer-related possibility, etc. of searched proteins were confirmed by investigating protein databases, including Swiss-Prot DB, NCBI nr DB, etc., related papers, and literatures and then, candidate glycoproteins expected to have at cancer-related possibility were selected. It can be confirmed that if selected, glycoproteins can be used for cancer markers or not, through a quantitative analysis by MRM MS method using hydrolyzed peptides which were derived from each protein as agents.

Table 1 below shows candidate marker peptide panel including tryptic peptides which can be concurrently generated by the hydrolysis with trypsin from one glycoprotein selected as a cancer marker candidate. In one embodiment, the peptides generated with trypsin are used for the hydrolysis of representatively screened glycoproteins. However, in theory, it is also possible to construes a candidate marker peptide panel using the peptides generated from the same glycoprotein using hydrolases other than trypsin, such as, for example, arginin C (Arg-C), aspartic acid N (Asp-N), glutamic acid C (Glu-C), lysine C (Lys-C), chymotrypsin, etc.

TABLE 1

| SEQ | | |
|---|---|---|
| ID | Peptide | |
| NO | mass (Da) | Peptide sequence |
| 1 | 685.4 | IVDLVK |
| 2 | 749.4 | FLEDVK |
| 3 | 778.4 | SPLFMGK |
| 4 | 784.4 | VVNPTQK |
| 5 | 851.5 | SASLHLPK |
| 6 | 887.5 | AVLTIDEK |
| 7 | 921.4 | FLENEDR |
| 8 | 1007.5 | QIHDYVEK |
| 9 | 1014.6 | SVLGQLGITK |
| 10 | 1057.5 | EDPQGDAAQK |
| 11 | 1075.6 | LSSWVLLMK |
| 12 | 1089.6 | WERPFEVK |
| 13 | 1109.6 | LSITGTYDLK |
| 14 | 1189.6 | LGMFNIQHCK |
| 15 | 1575.8 | DTVFALVNYIFFK |
| 16 | 1640.9 | ITPNLAEFAFSLYR |
| 17 | 1754.9 | YLGNATAIFFLPDEGK |
| 18 | 1778.8 | TDTSHHDQDHPTFNK |
| 19 | 1803.0 | LQHLENELTHDIITK |
| 20 | 1832.9 | VFSNGADLSGVTEEAPLK |
| 21 | 1855.0 | FNKPFVFLMIEQNTK |
| 22 | 1890.8 | DTEEEDFHVDQVTTVK |
| 23 | 2056.9 | LYHSEAFTVNFGDTEEAK |
| 24 | 2258.1 | GTEAAGAMFLEAIPMSIPPEVK |
| 25 | 2573.3 | TLNQPDSQLQLTTGNGLFLSEGLK |
| 26 | 3180.6 | QLAHQSNSTNIFFSPVSIATAFAMLSLGTK |
| 27 | 3690.3 | ADTHDEILEGLNFNLTEIPEAQIHEGFQELLR |

Since the marker peptides included in the above [Table 1] could be generated from one identical glycoprotein by hydrolysis, every peptide in the above [Table 1], alone or in combination can be theoretically used as an agent for a marker glycoprotein in a quantitative analysis. Further, if the other glycoprotein-originated peptides, which can be generated concurrently with the peptides of Table 1 from the other glycoproteins present in the same specimen, contain specific offsetting structure related to the cancer progression that has affinity to the lectin used, these other glycoprotein-originated peptides can also be used in combination with the peptides of Table 1 for the purpose of cancer diagnosis.

### EXAMPLE 3

### Identification of Marker Peptide Using Mass Analysis

For MPM quantification of the marker peptide of SEQ ID NO:9 (Peptide mass, 1014.6 (Da)) as a representative example among marker peptides of the above [Table 1], isotope-labeled standard of the peptide was prepared and added equally to respective peptide samples of pooled cancer plasma and pooled control plasma which were prepared in <Example 1> as an internal standard for a quantitative analysis. The LC/MRM quantitative mass analysis on the marker peptide of SEQ ID NO:9 was repeatedly quantified for each sample.

Consequently, as shown in FIG. 1, the marker peptide of SEQ ID NO:9 was quantitated at a level of around 34.7 fmol in pooled cancer plasma and at a level of around 16.4 fmol in the same amount of pooled control plasma. That is, it was confirmed that the marker peptide of SEQ ID NO:9 of the HOC patient group was quantitated approximately 2.1 times greater than that of the normal control group (FIG. 1).

### EXAMPLE 4

### Verification of Waiver Peptides Using Mass Analysis

According to the method of the present invention, the verification of peptide markers of [Table 1] was conducted with real blood samples of hepatocellular carcinoma patients and people having no cancer-related findings.

To be specific, MRM quantitative analysis was conducted repeatedly by three times with respect to the marker peptide with SEQ ID NO: 9 selected as a representative example from Table 1, using ten blood, specimens of clinically-confirmed patient with hepatocellular carcinoma (HCC), and thirty control blood specimens with no diagnosis of cancer. In addition to the marker peptide of SEQ ID NO: 9, MRM quantitative analysis was also conducted with respect to a combination of the marker peptide of SEQ ID NO: 9 and the peptides also generated by the hydrolysis performed in the process of preparing specimens, using alpha-1-acid glycoprotein 1 (A1AG1), alpha-1-antichymotrypsin (AACT), and ceruloplasmin (CERU). The thirty control blood specimens consisted of ten specimens diagnosed of hepatitis B-infected, ten specimens diagnosed of hepatitis B-infected/cirrhosis, and ten normal specimens with no diagnosis of the above two. The analysis data on the total forth specimens was obtained by the box-and-whisker plot, based on the normalization of the median values of the thirty control bloods.

Referring to FIG. 2, as a result of analysis on SEQ ID NO: 9, SEQ ID NO: 9 was measured to be the quantitative values that are higher than the thirty control bloods of the blood specimens of the ten liver cancer patients. Further, FIG. 3 represents a receiver operating characteristic (ROC) curve representing a result of conducting MRM quantitative analysis on the target peptide of SEQ ID NO: 9, in which the ten liver cancer blood specimens and thirty control blood specimens are used. As a result, with the area under ROC (AUROC)=0.92, the marker specificity was 86.7% at the sensitivity of 90%. From the above finding, it was verified that it is possible to differentiate normal group from liver cancer patients based on the analysis on the blood specimens, by using the marker peptide of SEQ ID NO: 9.

Further, with the method of using peptide marker according to an embodiment, reliability of the developed marker glycoproteins will certainly be increased, if one or more marker peptides listed in Table 1 provided above, which can be generated with the hydrolysis of the same marker glycoproteins, are used in combination for the purpose of quantitative mass spectrometry. Furthermore, the MRM quantitative analysis on the peptides generated together from the other glycoproteins in the blood specimen in the process of producing marker peptide with SEQ ID NO: 9, which are, alpha-1-acid glycoprotein 1 (A1AG1), alpha-1-antichymotrypsin (AACT), and ceruloplasmin (CERU), was conducted along with the analysis on the marker peptide with SEQ ID NO: 9. That is, peptide TEDTIFLR with SEQ ID NO: 28 as a representative example of the tryptic peptides generated with hydrolysis of A1AG1, peptide NLAVSQVVHK with SEQ ID NO: 29 as a representative example of tryptic peptides generated with the hydrolysis of AACT, and peptide GAYPLSIEPIGVR with SEQ ID NO: 30 as a representative example of tryptic peptides generated with the hydrolysis of CERU, were selected and combined with peptide with SEQ ID NO: 9 and then went through quantitative analysis. As a result, it was confirmed that the peptides originated from the glycoproteins such as A1AG1, AACT, and CERU can also make marker peptides which can distinguish liver cancer specimens from the control specimens (see Table 4). It is very certain that the other peptides, which are generated in the process of hydrolysis from the same marker glycoprotein, can also be used as the marker peptide, like the peptides that are representatively used. Additionally, the result of quantitative analysis on the three peptides TEDTIFLR, NLAVSQVVHK, GAYPLSIEPIGVR (SEQ IDS NOS: 28, 29, 30) as the representative examples of additional markers, were combined with the result of quantitative analysis on the marker peptide with SEQ ID NO; 9 using the artificial neural network algorithm, and the resultant data was statistically processed. FIG. 5 shows a ROC curve obtained as a result of combining the quantitative analysis results on the four marker peptides and statistically processing the combined data, in which the quantitative analysis results were obtained with respect to the ten liver cancer blood specimens and thirty blood specimens as a control. As a result, it was confirmed that the area under ROC (AUROC) obtained from the multi-markers based on the combination of the four individual peptide markers was 0.95, which was improved from the AUROC (0.92) obtained based on a single marker with SEQ ID NO: 9. From the above finding, it was verified that it is possible to distinguish between normal group and patients with liver cancer based on the analysis on blood specimens, using the marker peptide with SEQ ID NO: 9. It was also verified that, by additionally using the peptides originated from the glycoproteins such as A1AG1, AACT, and CERU as the multi-markers, it is possible to further increase the reliability of the method for distinguishing liver cancer specimens from the control specimens with no diagnosed liver cancer (see FIG. 5).

The foregoing exemplary embodiments and advantages are merely exemplary and are not to be construed as limiting the present invention. The present teaching can be readily applied to other types of apparatuses. Also, the description of the exemplary embodiments of the present inventive concept is intended to be illustrative, and not to limit the scope or the claims.

What is claimed is:

1. A method for a sequence analysis or a quantitative analysis of polypeptide for providing information for liver cancer diagnosis, the method comprising the step of:
1) isolating glycoprotein and concentrating the same by treating a specimen originated from a subject with lectin;
2) preparing polypeptide by hydrolyzing the glycoprotein of step 1);
3) performing a sequence analysis or quantitative analysis on the polypeptide of step 2); and
4) determining the subject to be with a high possibility of having liver cancer or be with liver cancer, if the result of the sequence analysis and quantitative analyses of step 3) indicate presence of polypeptide with an amino acid sequence of SEQ ID NOS: 1 to 27.

2. The method of claim 1, wherein the sequence analysis and the quantitative analysis on the polypeptide of step 3) are performed along with the quantitative analysis on the polypeptides isolated by hydrolysis of one or more glycoproteins selected from the group consisting of alpha-1-acid glycoprotein 1 (A1AG1), alpha-1-antichymotrypsin (AACT), and ceruloplasmin (CERU).

3. The method of claim 1, wherein the specimen of step 1) is any one selected from the group consisting of cell, cell culture medium, blood, serum, and plasma.

4. The method of claim 1, wherein the lectin of step 1) is a combination of one or more selected, from the group consisting of ConA, WGA, Jacalin, SNA, AAL, L-PHA, PNA, LCA, ABA, DBA, DSA, EGA, SBA, SSA, UEA, VVL and BPL.

5. The method of claim 1, wherein the hydrolysis of step 2) uses an enzyme selected from the group consisting of arginin C (Arg-C), aspartic acid N (Asp-N), glytamic acid C (Glu-C), lysine C (Lys-C), chymotrypsin, and trypsin.

6. The method of claim 1, wherein the quantitative analysis of step 3) is performed using liquid chromatography-mass spectrometry (LC-MS).

7. The method of claim 1, prior to the quantitative analysis of step 3), further comprising a step of concentrating using an antibody against the peptide of step 2).

8. The method of claim 1, wherein the quantitative analysis of step (3) uses a polypeptide for a standard material, the polypeptide having the same amino acid sequence as any one of polypeptide sequences of SEQ ID NOs: 1 to 27 and being labeled with an isotope of a different mass.

9. The method of claim 1, wherein the quantitative analysis of step (3) uses an antibody or a combination of antibodies which binds specifically to a polypeptide having any one of amino acid sequences of SEQ ID NOs:1 to 77.

10. The method of claim 1, wherein the quantitative analysis of step (3) uses a biochip for cancer diagnosis, the biochip comprising biomolecules that are capable of binding specifically to a polypeptide having any one of amino acid sequences of SEQ ID NOs:1 to 27 and are accumulated on a solid substrate.

11. The method of claim 14, wherein the biomolecules are an antibody or an aptamer.

12. The method of claim 1, wherein the polypeptide with an amino acid sequence of SEQ ID NO: 1 has 685.4 of molecular weight;
molecular weight of the polypeptide with amino acid sequence of SEQ ID NO: 2 is 749.4;
molecular weight of the polypeptide with amino acid sequence of SEQ ID NO:3 is 778.4;
molecular weight of the polypeptide with amino acid sequence of SEQ ID NO: 4 is 784.4;
molecular weight of the polypeptide with amino acid sequence of SEQ ID NO: 5 is 851.5;
molecular weight of the polypeptide with amino acid sequence of SEQ ID NO: 6 is 887.5;
molecular weight of the polypeptide with amino acid sequence of SEQ ID NO:7 is 921.4;
molecular weight of the polypeptide with amino acid sequence of SEQ ID NO: 8is 1007.5;
molecular weight of the polypeptide with amino acid sequence of SEQ ID NO: 9 is 1014.6;
molecular weight of the polypeptide with amino acid sequence of SEQ ID NO: 10 is 1057.5;
molecular weight of the polypeptide with amino acid sequence of SEQ ID NO: 11 is 1075.6;
molecular weight of the polypeptide with amino acid sequence of SEQ ID NO: 12 is 1089.6;
molecular weight of the polypeptide with amino acid sequence of SEQ ID NO: 13 is 1109.6;
molecular weight of the polypeptide with amino acid sequence of SEQ ID NO: 14 is 1189.6;
molecular weight of the polypeptide with amino acid sequence of SEQ ID NO: 15 is 1575.8;
molecular weight of the polypeptide with amino acid sequence of SEQ ID NO: 16 is 1640.9;
molecular weight of the polypeptide with amino acid sequence of SEQ ID NO: 17 is 1754.9;
molecular weight of the polypeptide with amino acid sequence of SEQ ID NO: 18 is 1778.8;
molecular weight of the polypeptide with amino acid sequence of SEQ ID NO: 19 is 1803.0;
molecular weight of the polypeptide with amino acid sequence of SEQ ID NO: 20 is 1832.9;
molecular weight of the polypeptide with amino acid sequence of SEQ ID NO: 21 is 1855.0;
molecular weight of the polypeptide with amino acid sequence of SEQ ID NO: 22 is 1890.8;
molecular weight of the polypeptide with amino acid sequence of SEQ ID NO: 23 is 2050.9;
molecular weight of the polypeptide with amino acid sequence of SEQ ID NO: 24 is 2258.1;
molecular weight of the polypeptide with amino acid sequence of SEQ ID NO: 25 is 2573.3;
molecular weight of the polypeptide with amino acid sequence of SEQ ID NO: 26 is 3180.6; and
molecular weight of the polypeptide with amino acid sequence of SEQ ID NO: 27 is 3690.8.

13. The method of claim 2, wherein the polypeptide isolated by the hydrolysis of alpha-1-acid glycoprotein 1 (A1AG1) has an amino acid sequence expressed by SEQ ID NO: 28, the polypeptide isolated by the hydrolysis of alpha-1-antichymotrypsin (AACT) has an amino acid sequence expressed by SEQ ID NO: 29, and the polypeptide isolated by the hydrolysis of ceruloplasmin (CERU) has an amino acid sequence expressed by SEQ ID NO: 30.

14. A method of quantitatively analyzing polypeptide to provide information for the diagnosis of liver cancer, the method comprising:
1) isolating and concentration glycoprotein by treating a specimen originated from a subject with lectin;
2) preparing polypeptide by hydrolyzing the glycoprotein of step 1);
3) quantitatively analyzing the polypeptide of step 2); and
4) determining the subject to be with a high risk of having liver cancer, or be with liver cancer, if a result of the quantitative analysis of step 3) indicates a presence of polypeptide having a molecular weight selected from the group consisting of 685.1, 749.4, 778.4, 784.4, 851.5, 887.5, 921.4, 1007.5, 1014.6, 1057.5, 1075.6, 1089.5, 1109.6, 1189.6, 1575.8, 1640.9, 1754.9, 1778.8, 1803.0, 1832.9, 1855.0, 1890.8, 2056.9; 2258.1, 2573.3, 3180.6, and 3690.8.

15. The method of claim 14, wherein the quantitative analysis on polypeptide of step 3) is performed along with a quantitative analysis on polypeptide which is isolated by hydrolysis of one or more glycoproteins selected from the group consisting of alpha-1-acid glycoprotein 1 (A1AG1), alpha-1-antichymotrypsin (AACT), and ceruloplasmin (CERU).

16. The method of claim 15, wherein the polypeptide isolated by the hydrolysis of alpha-1-acid glycoprotein 1 (A1AG1) has an amino acid sequence expressed by SEQ ID NO: 28, the polypeptide isolated by the hydrolysis of alpha-1-antichymotrypsin (AACT) has an amino acid sequence expressed by SEQ ID NO: 29, and the polypeptide isolated by the hydrolysis of ceruloplasmin (CERU) has an amino acid sequence expressed by SEQ ID NO: 30.


SEQUENCE LISTING (30 sequences)

SEQ ID NO: 1 - protein, 6 aa, Homo sapiens
IVDLVK

SEQ ID NO: 2 - protein, 6 aa, Homo sapiens
FLEDVK

SEQ ID NO: 3 - protein, 7 aa, Homo sapiens
SPLFMGK

SEQ ID NO: 4 - protein, 7 aa, Homo sapiens
VVNPTQK

SEQ ID NO: 5 - protein, 8 aa, Homo sapiens
SASLHLPK

SEQ ID NO: 6 - protein, 8 aa, Homo sapiens
AVLTIDEK

SEQ ID NO: 7 - protein, 7 aa, Homo sapiens
FLENEDR

SEQ ID NO: 8 - protein, 8 aa, Homo sapiens
QINDYVEK

SEQ ID NO: 9 - protein, 10 aa, Homo sapiens
SVLGQLGITK

SEQ ID NO: 10 - protein, 10 aa, Homo sapiens
EDPQGDAAQK

SEQ ID NO: 11 - protein, 9 aa, Homo sapiens
LSSWVLLMK

SEQ ID NO: 12 - protein, 8 aa, Homo sapiens
WERPFEVK

SEQ ID NO: 13 - protein, 10 aa, Homo sapiens
LSITGTYDLK

SEQ ID NO: 14 - protein, 10 aa, Homo sapiens
LGMFNIQHCK

SEQ ID NO: 15 - protein, 13 aa, Homo sapiens
DTVFALVNYIFFK

SEQ ID NO: 16 - protein, 14 aa, Homo sapiens
ITPNLAEFAFSLYR

SEQ ID NO: 17 - protein, 16 aa, Homo sapiens
YLGNATAIFFLPDEGK

SEQ ID NO: 18 - protein, 15 aa, Homo sapiens
TDTSHHDQDHPTFNK

SEQ ID NO: 19 - protein, 15 aa, Homo sapiens
LQHLENELTHDIITK

SEQ ID NO: 20 - protein, 18 aa, Homo sapiens
VFSNGADLSGVTEEAPLK

SEQ ID NO: 21 - protein, 15 aa, Homo sapiens
FNKPFVFLMIEQNTK

SEQ ID NO: 22 - protein, 16 aa, Homo sapiens
DTEEEDFHVDQVTTVK

SEQ ID NO: 23 - protein, 18 aa, Homo sapiens
LYHSEAFTVNFGDTEEAK

SEQ ID NO: 24 - protein, 22 aa, Homo sapiens
GTEAAGAMFLEAIPMSIPPEVK

SEQ ID NO: 25 - protein, 24 aa, Homo sapiens
TLNQPDSQLQLTTGNGLFLSEGLK

SEQ ID NO: 26 - protein, 30 aa, Homo sapiens
QLAHQSNSTNIFFSPVSIATAFAMLSLGTK

SEQ ID NO: 27 - protein, 32 aa, Homo sapiens
ADTHDEILEGLNFNLTEIPEAQIHEGFQELLR

SEQ ID NO: 28 - protein, 8 aa, Homo sapiens
TEDTIFLR

SEQ ID NO: 29 - protein, 10 aa, Homo sapiens
NLAVSQVVHK

SEQ ID NO: 30 - protein, 13 aa, Homo sapiens
GAYPLSIEPIGVR